(19) United States
(12) Patent Application Publication
(10) Pub. No.: US 2026/0083876 A1
(43) Pub. Date: Mar. 26, 2026

(54) BIODEGRADABLE POLYMER SCAFFOLD COMPRISING DRUG AND/OR EXTRACELLULAR VESICLES AND METHOD FOR PREPARING SAME

(71) Applicant: CHA UNIVERSITY INDUSTRY-ACADEMIC COOPERATION FOUNDATION, Pocheon-si Gyeonggi-do (KR)

(72) Inventors: Dong Keun HAN, Seoul (KR); Seung Yeon LEE, Anyang-si (KR); Won-Kyu RHIM, Seongnam-si (KR)

(73) Assignee: CHA UNIVERSITY INDUSTRY-ACADEMIC COOPERATION FOUNDATION, Pocheon-si Gyeonggi-do (KR)

(21) Appl. No.: 19/402,801

(22) Filed: Nov. 26, 2025

Related U.S. Application Data
(63) Continuation of application No. PCT/KR2024/096867, filed on Dec. 12, 2024.

(30) Foreign Application Priority Data
Dec. 13, 2023 (KR) 10-2023-0181083

(51) Int. Cl. A61L 27/04; A61L 27/10; A61L 27/36; A61L 27/54; A61L 27/58

(52) U.S. Cl. CPC A61L 27/047; A61L 27/10; A61L 27/3633; A61L 27/54; A61L 27/58; A61L 2430/26

(57) ABSTRACT

An aspect provides a biodegradable polymer scaffold for kidney regeneration, including basic ceramic particles, an extracellular matrix, zinc particles, a kidney regeneration-inducing material, and a biodegradable polymer. A biodegradable polymer scaffold for kidney regeneration, according to an aspect, includes a kidney regeneration-inducing material and/or extracellular vesicles that secrete a stem cell recruitment-inducing factor, thereby inducing stem cells to a damaged tissue site and enhancing kidney regenerative capacity, and thus can effectively induce the regeneration of kidney tissue. Therefore, the biodegradable polymer scaffold can contribute to the medical device industry, including the bioimplant market.

### CROSS-REFERENCE TO RELATED APPLICATIONS

This application is a continuation of International Application No. PCT/KR2024/096867 filed on Dec. 12, 2024, which claims priority to Korean Patent Application No. 10-2023-0181083 filed on Dec. 13, 2023, the entire contents of which are herein incorporated by reference.

### TECHNICAL FIELD

The present disclosure relates to a biodegradable polymer scaffold containing a drug for inducing kidney tissue regeneration and/or extracellular vesicles that secrete a stem cell recruitment-inducing factor, and a method of preparing the same.

### BACKGROUND ART

The kidneys are two bean-shaped organs that filter blood and are involved in controlling the volume of various body fluids, osmotic concentration of body fluids, acid-base balance, concentration control of various electrolytes, excretion of waste products, and the like. The nephron is the functional unit of the kidney and consists of a glomerulus and a tubule. The glomerulus is a structure consisting of tiny capillaries tangled together like a ball of yarn and serves to filter waste products from the blood, while the tubule is a thin tube approximately 4 to 7 cm long that reabsorbs filtered substances and concentrates waste products to form urine. The kidneys are essential organs for maintaining homeostasis in the body, and the occurrence of functional impairment has serious effects on various parts of the body depending on the severity.

Tissue engineering is a new field that has emerged with scientific advancement, and is a multidisciplinary field that integrates and applies basic concepts and scientific technologies from life sciences, engineering, medicine, and the like. Tissue engineering is an applied science that aims to understand the correlation between the structure and function of biological tissues, and ultimately to maintain, enhance or restore the body functions by creating artificial tissues that can be implanted into the body to replace damaged tissues or organs with normal tissues or regenerate the damaged tissues or organs.

Representative tissue engineering techniques are summarized as follows. First, necessary tissue is harvested from the body of a patient, cells are isolated from pieces thereof, and then the isolated cells are cultured for proliferation in a required amount. The proliferated cells are seeded onto a polymer scaffold and cultured in vitro for a certain period of time, and the resulting hybrid cell/polymer structure is then reimplanted into the human body. This technique involves applying a method in which the implanted cells initially receive oxygen and nutrients through diffusion of body fluids until new blood vessels form in tissues or organs, and once blood vessels grow in the body and blood is supplied, the cells proliferate and differentiate to form new tissues and organs, while the polymer scaffold is degraded and eventually disappears during this process.

Therefore, the polymer scaffold must secure sufficient requirements and functions for tissue regeneration in the human body. Materials for tissue regeneration are required to have cell affinity to: enable tissue cells to adhere to the surface of a material, thereby forming new blood vessels; and form three-dimensional tissue structures, and to function as an intermediate barrier positioned between implanted cells and host cells. This means that the material must be non-toxic and biocompatible such that no blood coagulation or inflammatory response occurs after implantation. In addition, during the regeneration process, it is desirable that numerous cells are recruited to and mixed with the scaffold or defect site. Therefore, during a cell mixing process in the scaffold, chemotactic signaling molecules, which are commonly referred to as chemokines, are typically used to enhance initial cell recruitment.

Chemokine stromal derived factor-1α (SDF-1α) is attracting attention because SDF-1α is chemotactic for mesenchymal stem cells (MSCs) and promotes the expression of genes related to cell motility. This also increases cell regeneration by recruiting mesenchymal stem cells to an injury site and guides the cells into the scaffold. Thus, scaffolds capable of recruiting cells after implantation must be designed to release signaling molecules over a certain period of time, and also require other additional factors related to regeneration, providing an appropriate environment for kidney regeneration.

Therefore, there is a need to develop a multifunctional composite scaffold that can effectively induce regeneration of kidney tissue by inducing stem cells to a damaged tissue site and enhancing kidney regenerative capacity.

### DISCLOSURE OF INVENTION

### Technical Problem

An aspect provides a biodegradable polymer scaffold for kidney regeneration, including basic ceramic particles, an extracellular matrix, zinc particles, a kidney regeneration-inducing material, and a biodegradable polymer.

Another aspect provides a bioimplant for kidney regeneration, including the biodegradable polymer scaffold.

Another aspect provides a method of preparing a biodegradable polymer scaffold, the method including preparing a first polymer solution by mixing basic ceramic particles, an extracellular matrix, zinc particles, and a biodegradable polymer, preparing a second polymer solution by mixing the first polymer solution and a porogen, and drying the second polymer solution to prepare a porous scaffold, and the method further including, in the preparation of the first polymer solution, mixing together with a kidney regeneration-inducing material, or, after the preparation of the porous scaffold, loading the kidney regeneration-inducing material.

### Solution to Problem

An aspect relates to a biodegradable polymer scaffold for kidney regeneration, including basic ceramic particles, an extracellular matrix, zinc particles, a kidney regeneration-inducing material, and a biodegradable polymer.

The biodegradable polymer scaffold can not only adjust the pore size, density, porosity and the like of a porous polymer scaffold, but can also adjust, in various ways, the shape and size of a polymer scaffold including an extracellular matrix and a kidney regeneration-inducing material (e.g., extracellular vesicles or a kidney regeneration-inducing drug). Also, the biodegradable polymer scaffold may contain basic ceramic particles, an extracellular matrix, zinc particles, and a kidney regeneration-inducing material (e.g., extracellular vesicles or a kidney regeneration-inducing drug) at various concentrations, thereby exhibiting enhanced tissue regenerative capacity, improved hydrophilicity, and controlled mechanical strength and degradation period.

According to an aspect, the basic ceramic particles may be one or more selected from the group consisting of an alkali metal, an oxide of the alkali metal, a hydroxide of the alkali metal, an alkaline earth metal, an oxide of the alkaline earth metal, and a hydroxide of the alkaline earth metal. The alkali metal or alkaline earth metal may be, for example, lithium (Li), beryllium (Be), sodium (Na), magnesium (Mg), potassium (K), calcium (Ca), rubidium (Rb), strontium (Sr), barium (Ba), cesium (Cs), francium (Fr), radium (Ra), and the like.

In an aspect, the oxide of the alkali metal, the hydroxide of the alkali metal, the oxide of the alkaline earth metal, or the hydroxide of the alkaline earth metal may be selected from the group consisting of lithium hydroxide, beryllium hydroxide, sodium hydroxide, magnesium hydroxide, potassium hydroxide, calcium hydroxide, rubidium hydroxide, strontium hydroxide, barium hydroxide, cesium hydroxide, francium hydroxide, radium hydroxide, magnesium oxide, sodium oxide, lithium oxide, sodium oxide, manganese oxide, potassium oxide, calcium oxide, barium oxide, cesium oxide, and radium oxide.

In an aspect, the basic ceramic particles may be surface-modified with a fatty acid, a polymer material, or a mixture thereof.

The fatty acid may be, for example, caprylic acid, capric acid, lauric acid, myristoleic acid, palmitoleic acid, sapienic acid, oleic acid, elaidic acid, vaccenic acid, linoleic acid, ricinoleic acid, linoelaidic acid, α-linoleic acid, arachidonic acid, eicosapentaenoic acid, erucic acid, stearic acid, DHA, octadecanoic acid, coconut oil, palm oil, cotton seed oil, wheat germ oil, bean oil, olive oil, corn oil, sunflower oil, safflower oil, hampseed oil, canola oil, or the like.

In other embodiments, the polymer material may be, for example, one or more monomers selected from the group consisting of L-lactide, D-lactide, D,L-lactide, glycolide, caprolactone, dioxanone, trimethylene carbonate, hydroxyalkanoate, peptide, cyanoacrylate, lactic acid, glycolic acid, hydroxycaproic acid, maleic acid, phosphazene, amino acids, hydroxybutyric acid, sebacic acid, hydroxyethoxyacetic acid, and trimethylene glycol, or may be one prepared therefrom, e.g., poly-L-lactide, poly-D-lactide, poly-D,L-lactide, polyglycolide, polycaprolactone, poly-L-lactide-co-glycolide, poly-D-lactide-co-glycolide, poly-D,L-lactide-co-glycolide, poly-L-lactide-co-caprolactone, poly-D-lactide-co-caprolactone, poly-D,L-lactide-co-caprolactone, polyglycolide-co-caprolactone, polydioxanone, polytrimethylene carbonate, polyglycolide-co-dioxanone, polyamide ester, polypeptide, polyorthoester, polymaleic acid, polyphosphazene, polyanhydride, polysebacic anhydride, polyhydroxyalkanoate, polyhydroxybutyrate, polycyanoacrylate, or the like.

The term “surface modification” refers to changing the chemical and physical structures of a particle surface, for example, causing structural changes by introducing various functional groups to the surface of a basic ceramic particle by using a biodegradable polymer.

A biodegradable polymer scaffold including basic ceramic particles that are surface-modified with a fatty acid or polymer material, according to an aspect, may have improved dispersion stability in an organic solvent and improved mechanical properties, and acidic substances may be neutralized with basic ceramic particles, thereby improving inflammatory responses and cytotoxicity in the body.

According to an aspect, the basic ceramic particles or surface-modified basic ceramic particles may have a diameter (size) of 1 nm to 1 mm. In case that the diameter of the basic ceramic particles exceeds the above range, precipitation occurs due to the weight of the basic ceramic particles, resulting in phase separation in an organic solvent.

According to an aspect, the basic ceramic particles may be comprised in an amount of 1 to 20 wt % with respect to a total weight of the biodegradable polymer scaffold. The basic ceramic particles may be comprised in an amount of, for example, 1 to 20 wt %, 1 to 16 wt %, 1 to 12 wt %, 5 to 20 wt %, 5 to 16 wt %, 5 to 12 wt %, 10 to 20 wt %, 10 to 16 wt %, or 10 to 12 wt %, with respect to the total weight of the biodegradable polymer scaffold. In case that the amount of the basic ceramic particles is less than 1 wt %, an acidic substance, which is a degradation product of the polymer scaffold, cannot be sufficiently neutralized. In case that the amount of the basic ceramic particles exceeds 20 wt %, alkalization of the environment around the polymer scaffold may be induced.

In an aspect, the extracellular matrix may be isolated from a human or animal.

Specifically, the extracellular matrix is a matrix protein derived from tissues or cells of a human or animal, and may be in a complexly mixed state or in an artificially isolated single molecular state, and the protein may have a denatured structure. The extracellular matrix may be derived from, for example, vertebrates such as humans, pigs, cows, rats, sheep, horses, dogs, or cats, and may be isolated from bone, kidney, amniotic membrane, skin, brain, small intestinal submucosa, fascia, or spinal cord meninges, depending on the purpose. The extracellular matrix may be appropriately selected according to the structure or function thereof. In an aspect, the extracellular matrix may be a group having a fibrous structure, a group related to bone differentiation and osteogenesis, a glycosaminoglycan group, a proteoglycan group, or the like. The group having a fibrous structure may be, for example, collagen fibers, elastin fibers, laminin, fibrinogen, fibronectin, gelatin, or the like. The collagen may be type I, II, III, IV, V, VI, VII, VIII, IX, X, XI, XII, XIV, XV, XVI, XVIII, XIX, XX, XXI, XXII, XXIII, XXIV, XXV, XXVI, XXVII, XXVIII collagen, or the like. In other embodiments, the group related to bone differentiation and osteogenesis may be, for example, osteonectin, osteopontin, vitronectin, vimentin, or the like. In other embodiments, the glycosaminoglycan group, may be, for example, heparan sulfate, keratan sulfate, chondroitin sulfate, dermatan sulfate, heparin, low-molecular-weight heparin, hyaluronic acid, or the like. In other embodiments, the proteoglycan group may be, for example, decorin, biglycan, versican, tertican, perlecan, bikunin, neurocan, aggrecan, fibromodulin, lumican, or the like.

In an aspect, the extracellular matrix may be comprised in an amount of 10 to 50 wt % with respect to the total weight of the biodegradable polymer scaffold. The extracellular matrix may be comprised in an amount of, for example, 10 to 50 wt %, 10 to 40 wt %, 10 to 35 wt %, 20 to 50 wt %, 20 to 40 wt %, 20 to 35 wt %, 25 to 50 wt %, 25 to 40 wt %, or 25 to 35 wt %, with respect to the total weight of the biodegradable polymer scaffold. In case that the amount of the extracellular matrix is less than 10 wt %, the effect of improving cell compatibility cannot be sufficiently exhibited. In case that the amount of the extracellular matrix exceeds 50 wt %, the mechanical properties of the polymer scaffold decrease.

In another aspect, the extracellular matrix may be decellularized, and may be decellularized by a physical or chemical method after tissue or cell culture. The physical decellularization method may include, for example, freeze-thawing, ultrasonication, or physical stirring, and the chemical decellularization method may include, for example, treatment of animal-derived tissue powder with a hypotonic solution containing water, an anionic surfactant, a non-ionic surfactant, cationic surfactant, DNase, RNase, trypsin, or the like. In the chemical decellularization method, a Tris-HCl (pH 8.0) solution may be used as the hypotonic solution, and sodium dodecyl sulfate (SDS), sodium deoxycholate, Triton X-200, or the like may be used as the anionic surfactant. In other embodiments, Triton X-100, Tween 20, or Tween 80 may be used as the non-ionic surfactant, and CHAPS, sulfobetaine-10 (SB-10), sulfobetaine-16 (SB-16), tri-n-butyl phosphate, N-lauroyl-sarcosinate, IGEPAL CA-630, or the like may be used as the cationic surfactant. In other embodiments, the decellularization may be performed before or after performing a powdering process after collection of animal tissue, e.g., kidney tissue, or may be performed simultaneously with the powdering process.

In an aspect, the zinc particles may be one or more selected from the group consisting of zinc oxide, zinc sulfide, zinc nitrate, zinc selenide, zinc telluride, zinc nitride, zinc phosphide, zinc arsenide, zinc antimonide, zinc peroxide, zinc hydride, zinc oxalate dihydrate, zinc chloride, zinc bromide, zinc iodide, zinc hydroxide, zinc chlorate, zinc sulfate, zinc phosphate, zinc molybdate, zinc cyanide, zinc metaarsenite, zinc arsenate octahydrate, zinc chromate, zinc pyrithione, and zinc acetate.

In an aspect, the zinc particles may be surface-modified with a fatty acid, a polymeric material, or a mixture thereof.

The fatty acid may be, for example, caprylic acid, capric acid, lauric acid, myristoleic acid, palmitoleic acid, sapienic acid, oleic acid, elaidic acid, vaccenic acid, linoleic acid, ricinoleic acid, linoelaidic acid, α-linoleic acid, arachidonic acid, eicosapentaenoic acid, erucic acid, stearic acid, DHA, octadecanoic acid, coconut oil, palm oil, cotton seed oil, wheat germ oil, bean oil, olive oil, corn oil, sunflower oil, safflower oil, hempseed oil, canola oil, or the like.

In other embodiments, the polymer material may be, for example, one or more monomers selected from the group consisting of L-lactide, D-lactide, D,L-lactide, glycolide, caprolactone, dioxanone, trimethylene carbonate, hydroxyalkanoate, peptide, cyanoacrylate, lactic acid, glycolic acid, hydroxycaproic acid, maleic acid, phosphazene, amino acids, hydroxybutyric acid, sebacic acid, hydroxyethoxyacetic acid, and trimethylene glycol, or may be one prepared therefrom, e.g., poly-L-lactide, poly-D-lactide, poly-D,L-lactide, polyglycolide, polycaprolactone, poly-L-lactide-co-glycolide, poly-D-lactide-co-glycolide, poly-D,L-lactide-co-glycolide, poly-L-lactide-co-caprolactone, poly-D-lactide-co-caprolactone, poly-D, L-lactide-co-caprolactone, polyglycolide-co-caprolactone, polydioxanone, polytrimethylene carbonate, polyglycolide-co-dioxanone, polyamide ester, polypeptide, polyorthoester, polymaleic acid, polyphosphazene, polyanhydride, polysebacic anhydride, polyhydroxyalkanoate, polyhydroxybutyrate, polycyanoacrylate, or the like.

In an aspect, the zinc particles or surface-modified zinc particles may have a diameter (size) of 10 to 1000 nm. Specifically, the zinc particles or surface-modified zinc particles may have a diameter of, for example, 10 to 1000 nm, 10 to 800 nm, or 50 to 400 nm.

In an aspect, the zinc particles may be comprised in an amount of 1 to 10 wt % with respect to the total weight of the biodegradable polymer scaffold. The zinc particles may be comprised in an amount of, for example, 1 to 10 wt %, 1 to 8 wt %, 1 to 7 wt %, 3 to 10 wt %, 3 to 8 wt %, 3 to 7 wt %, 4 to 10 wt %, 4 to 8 wt %, or 4 to 7 wt %, with respect to the total weight of the biodegradable polymer scaffold.

In an aspect, the kidney regeneration-inducing material may be edaravone (EDV), extracellular vesicles, or a mixture thereof.

Edaravone (chemical name: 3-methyl-1-phenyl-2-pyrazolin-5-one), which is a compound represented by Formula 1, is a free radical scavenger and a commercially available brain neuroprotective agent (Reference: Yakugaku Zasshi. 2004, 124(3): 99-111):

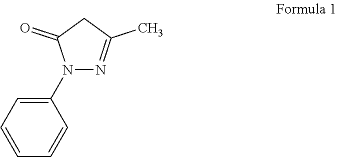

Previous studies revealed that edaravone has antioxidant activity and can significantly improve neurological deficits in cerebral ischemia-reperfusion animal models, reduce infarct size, decrease brain damage, alleviate brain edema, and inhibit lipid peroxidation in damaged brain tissue. Recent studies have confirmed that edaravone can protect against ischemia-reperfusion-induced kidney injury through JAK/STAT signaling, inhibit cell apoptosis, and improve mitochondrial damage (Reference: Zhao. 2020, 53(1): 28). It has also been confirmed that edaravone inhibits acute mitochondrial DNA damage, tubular epithelial cell death, and chronic kidney damage, which are caused by cisplatin, which rapidly damages mitochondrial DNA in the proximal tubules of the kidney and induces apoptosis of tubular epithelial cells, and thus has the effect of treating kidney damage (Reference: Iguchi. 2004, 38(4): 333-341). Therefore, it was confirmed that edaravone has a preventive or therapeutic effect on kidney damage and a kidney regeneration effect.

The term “prevention” may refer to all actions that inhibit or delay kidney-related diseases, including kidney damage in a subject, via administration.

The term “treatment” may refer to all actions that improve or beneficially alter symptoms of kidney-related diseases, including kidney damage in a subject, via administration.

The term “administration” refers to introducing a certain substance into a subject by using an appropriate method, and the term “subject” refers to all living organisms, such as rats, mice, and livestock, including humans, which may have kidney-related diseases, including kidney damage. Specifically, the subject may be, for example, mammals including humans.

The extracellular vesicles are nano-sized vesicles that all cells secrete into the external environment for information exchange between cells, and contain various substances that exhibit biological activity, such as proteins, lipids, nucleic acids, metabolites, and the like. The extracellular vesicles may include exosomes and microvesicles. For example, the extracellular vesicles may be isolated from stem cells derived from one or more selected from the group consisting of umbilical cord, umbilical cord blood, bone marrow, fat, muscle, skin, amniotic membrane, and placenta.

According to an aspect, the stem cells may be cells genetically engineered to overexpress a stem cell recruitment-inducing factor compared to parent cells, and the extracellular vesicles may secrete a stem cell recruitment-inducing factor. The stem cell recruitment-inducing factor may be, for example, stromal derived factor-1α (SDF-1α).

The term “parent cell” refers to a cell that has not been artificially manipulated to reduce the expression or activity of a gene encoding the stem cell recruitment-inducing factor or a stem cell recruitment-inducing factor protein, and refers to a cell freshly isolated from the human body and a cell cultured therefrom.

The term “genetic engineering” or “genetically engineered” refers to an act of introducing one or more genetic modifications into a cell or a cell produced thereby. The genetic engineering may be induced by modification in the nucleic acid sequence of a gene encoding the stem cell recruitment-inducing factor, and the modification in the nucleic acid sequence may be artificially caused, for example, by a CRISPR/Cas9 system.

SDF-1α is an unglycosylated protein containing 93 amino acids and having a molecular weight of 10.7 kDa, is a chemokine that plays a role in moving various immune cells to inflammatory synovium, and is a substance that is produced in vascular endothelial cells and serves to recruit vascular progenitor cells. That is, SDF-1α is known to be involved in angiogenesis. SDF-1α may be derived from a human, and according to an aspect, mesenchymal stem cells (MSCs) may be transformed to produce stem cell recruitment-inducing factor SDF-1α-secreting extracellular vesicles (SDF-1α EVs) capable of continuously expressing SDF-1α.

In an aspect, the biodegradable polymer may be one or more selected from the group consisting of polylactide, polyglycolide, polycaprolactone, polylactide-co-glycolide, polylactide-co-caprolactone, polyglycolide-co-caprolactone, polydioxanone, polytrimethylene carbonate, polyglycolide-co-dioxanone, polyamide ester, polypeptide, polyorthoester, polymaleic acid, polyanhydride, polysebacic anhydride, polyhydroxyalkanoate, polyhydroxybutyrate, and polycyanoacrylate.

According to an aspect, the biodegradable polymer may be comprised in an amount of 25 to 85 wt % with respect to the total weight of the biodegradable polymer scaffold. The biodegradable polymer may be comprised in an amount of, for example, 25 to 85 wt %, 25 to 75 wt %, 25 to 65 wt %, 35 to 85 wt %, 35 to 75 wt %, 35 to 65 wt %, 45 to 85 wt %, 45 to 75 wt %, or 45 to 65 wt %, with respect to the total weight of the biodegradable polymer scaffold.

According to an aspect, the biodegradable polymer may comprise 1 to 20 wt % of the basic ceramic particles, 10 to 50 wt % of the extracellular matrix, 1 to 10 wt % of the zinc particles, and 25 to 85 wt % of the biodegradable polymer with respect to the total weight of the biodegradable polymer scaffold.

In an aspect, in case that the kidney regeneration-inducing material comprises edaravone, the edaravone may be comprised in an amount of 0.1 to 1 wt % with respect to the total weight of the biodegradable polymer scaffold. In case that the kidney regeneration-inducing material includes edaravone, the kidney regeneration-inducing material may be comprised in an amount of, for example, 0.1 to 1 wt %, 0.1 to 0.8 wt %, 0.1 to 0.6 wt %, 0.2 to 1 wt %, 0.2 to 0.8 wt %, 0.2 to 0.6 wt %, 0.4 to 1 wt %, 0.4 to 0.8 wt %, or 0.4 to 0.6 wt %, with respect to the total weight of the biodegradable polymer scaffold.

In an aspect, in case that the kidney regeneration-inducing material comprises extracellular vesicles, 1×10⁶ to 1×10¹² of the extracellular vesicles may be comprised in the biodegradable polymer scaffold. In case that the kidney regeneration-inducing material comprises extracellular vesicles, the kidney regeneration-inducing material in the biodegradable polymer scaffold may be comprised in an amount of, for example, 1×10⁶ to 1×10¹², 1×10⁶ to 1×10¹¹, 1×10⁶ to 1×10¹⁰, 1×10⁷ to 1×10¹², 1×10⁷ to 1×10¹¹, 1×10⁷ to 1×10¹⁰, 1×10⁸ to 1×10¹², 1×10⁸ to 1×10¹¹, or 1×10⁸ to 1×10¹⁰.

Another aspect relates to a bioimplant for kidney regeneration, including the biodegradable polymer scaffold.

The “biodegradable polymer scaffold” and the like may be as described above.

In an aspect, the bioimplant may be used as a stent, a surgical suture, a tissue regeneration scaffold, a bio-nanofiber, a hydrogel, a bio-sponge, a pin, a screw, a rod, an implant, other cardiovascular-related materials, other dental materials, and other neurological/orthopedic/plastic surgical biomaterials.

Another aspect relates to a method of preparing a biodegradable polymer scaffold for kidney regeneration, the method including the steps of: preparing a first polymer solution by mixing basic ceramic particles, an extracellular matrix, zinc particles, and a biodegradable polymer;
preparing a second polymer solution by mixing the first polymer solution and a porogen; and
drying the second polymer solution to prepare a porous scaffold, and the method further including:
in the preparation of the first polymer solution, mixing together with a kidney regeneration-inducing material; or
after the preparation of the porous scaffold, loading the kidney regeneration-inducing material.

The “basic ceramic particles,” “extracellular matrix,” “zinc particles,” “biodegradable polymer,” “kidney regeneration-inducing material,” “biodegradable polymer scaffold,” “extracellular vesicles,” and the like may be as described above.

In an aspect, the preparation of the first polymer solution may include mixing the basic ceramic particles, the extracellular matrix, the zinc particles, and the biodegradable polymer in an organic solvent.

In an aspect, the organic solvent may be, for example: alcohols such as methanol, ethanol, propanol, and butanol; aldehydes such as ammonia, dimethyl sulfoxide, dimethylformamide, acetronitrile, tetrahydrofuran, formaldehyde, glutaraldehyde, and acetaldehyde; alkanes such as dioxane, chloroform, heptane, hexane, pentane, octane, nonane, and decane; benzene ring-type solvents such as benzene, toluene, and xylene; ethers such as ether, di-propyl ether, petroleum ether, and methyl-t-butyl ether; ketones such as propanone, butanone, pentanone, hexanone, and heptanone; and conventional organic solvents such as methylene chloride, tetrafluoroisopropane, and carbon tetrachloride.

In an aspect, the porogen may be ice particles, and the pore size and porosity of the porous polymer scaffold may be controlled by controlling the size and content of the ice particles. The ice particles may have a diameter (size) of 10 to 500 μm. The diameter of the ice particles may be, for example, in a range of 10 to 500 μm, 10 to 450 μm, 10 to 400 μm, 10 to 300 μm, 30 to 500 μm, 30 to 300 μm, 30 to 250 μm, 50 to 500 μm, 50 to 400 μm, 50 to 300 μm, 50 to 200 μm, 100 to 500 μm, 100 to 300 μm, or 150 to 200 μm. In case that the diameter of the ice particles is less than 10 μm, the cell infiltration rate is low, and the angiogenesis effect cannot be sufficiently exhibited. In case that the diameter of the ice particles exceeds 500 μm, the mechanical properties of the biodegradable polymer scaffold decrease.

In other embodiments, in the preparation of the second polymer solution, the porogen may be mixed in an amount of 100 to 2000 wt % with respect to a total weight of the first polymer solution. Specifically, the porogen may be mixed in an amount of 100 to 2000 wt %, 100 to 1500 wt %, 100 to 1300 wt %, 100 to 1000 wt %, 100 to 500 wt %, 500 to 2000 wt %, 500 to 1500 wt %, 500 to 1000 wt %, 1000 to 2000 wt %, or 1500 to 2000 wt %, with respect to the total weight of the first polymer solution. In case that the amount of the porogen mixed is less than 100 wt % with respect to the total weight of the first polymer solution, pores are not sufficiently formed inside the polymer scaffold. In case that the amount of the porogen mixed exceeds 2000 wt % with respect to the total weight of the first polymer solution, the mechanical properties of the polymer scaffold decrease.

In an aspect, the drying may be freeze-drying, and by preparing a polymer scaffold by the freeze-drying method, the detachment of basic ceramic particles and extracellular matrix that may occur in a conventional salt forming method may be prevented.

In other embodiments, specifically, in case that the kidney regeneration-inducing material used in the method is edaravone, the method may include mixing together with the kidney regeneration-inducing material in the preparation of the first polymer solution.

In case that extracellular vesicles are used as the kidney regeneration-inducing material in the method, the method may further include a step of loading a kidney regeneration-inducing material after the preparation of the porous scaffold.

Furthermore, in case that the kidney regeneration-inducing material used in the method is a mixture of edaravone and extracellular vesicles, the method may further include the steps of, in the preparation of the first polymer solution, mixing together with a kidney regeneration-inducing material, and after the preparation of the porous scaffold, loading extracellular vesicles.

### Advantageous Effects of Invention

A biodegradable polymer scaffold for kidney regeneration, according to an aspect, includes a kidney regeneration-inducing material and/or extracellular vesicles that secrete a stem cell recruitment-inducing factor, thereby inducing stem cells to a tissue damage site and enhancing kidney regenerative capacity, and thus can effectively induce the regeneration of kidney tissue. Therefore, the biodegradable polymer scaffold can contribute to the medical device industry, including the bioimplant market.

### BRIEF DESCRIPTION OF DRAWINGS

FIG. 1 schematically illustrates a polymer scaffold according to an aspect.

FIG. 2 is a vector map of an expression vector that can be used for the production of CRISPR-mediated SDF-1α-secreting extracellular vesicles (SDF-1α EVs).

FIG. 3 illustrates the immunoblotting results showing the expression level of SDF-1α-conjugated His in cell lysates.

FIG. 4 is a graph showing the degree of SDF-1α secretion in extracellular vesicles determined by ELISA.

FIG. 5 illustrates scanning electron microscope images acquired by analyzing the pore size and structure of a biodegradable polymer scaffold according to an aspect.

FIG. 6 illustrates the effect of a polymer scaffold according to an aspect on in vitro wound healing potency.

FIG. 7 illustrates the effect of a polymer scaffold according to an aspect on in vitro blood vessel formation.

### MODE FOR THE INVENTION

Hereinafter, the present disclosure will be described in more detail through examples. However, these examples are provided for illustrative purposes only and are not intended to limit the scope of the present disclosure.

### PREPARATION EXAMPLES

### Preparation Example 1. Preparation of Basic Ceramic Particles

### 1-1. Preparation of Magnesium Hydroxide Particles

10.8 g of sodium hydroxide was dissolved in 300 ml of triple-distilled water to prepare a sodium hydroxide solution. Subsequently, a magnesium nitrate solution prepared by dissolving 20 g of magnesium nitrate in 150 ml of triple-distilled water was added dropwise to the sodium hydroxide solution by using a dropping funnel at a rate of 40 drops per minute. The nano-magnesium hydroxide particles precipitated in the reaction solution were purified by washing with distilled water, and then collected by filtration. The obtained magnesium hydroxide particles were vacuum-dried and stored.

### 1-2. Preparation of Magnesium Oxide Particles

The magnesium hydroxide particles prepared in Preparation Example 1-1 were calcined at a temperature ranging from 500 to 1500° C. by using an electric furnace to prepare magnesium oxide particles.

1-3. Preparation of Magnesium Hydroxide Particles Surface-Modified with Polymer

The basic ceramic particles prepared in Preparation Example 1-1 were surface-modified with L-lactide. Specifically, 80 wt % of the magnesium hydroxide of Preparation Example 1-1 and 20 wt % of L-lactide were mixed with respect to a total weight of the mixture. Subsequently, 0.05 wt % of stannous octoate (catalyst) was diluted in toluene and added with respect to the total weight of the reactants (magnesium hydroxide and L-lactide). A glass reactor containing the reactants was kept under vacuum at 70° C. for 6 hours while stirring to completely remove toluene and moisture. A ring-opening polymerization reaction was performed for 48 hours while stirring the sealed glass reactor in an oil bath adjusted at 150° C. The recovered polymer was added to a sufficient amount of chloroform for more than 1 hour to remove homopolymer and unreacted residues, thereby preparing basic ceramic particles modified with the polymer.

1-4. Preparation of Magnesium Oxide Particles Surface-Modified with Polymer

Basic ceramic particles modified with a polymer were prepared in the same manner as in Preparation Example 1-3, except that the magnesium oxide particles prepared in Preparation Example 1-2 were used instead of the magnesium hydroxide particles.

### Preparation Example 2. Preparation of Decellularized and Powdered Extracellular Matrix

### 2-1. Preparation of Human Adipose-Derived Extracellular Matrix

Human adipose tissue was collected and washed three times with a physiological saline solution for 10 minutes each. The washed tissue was dehydrated with ethanol, and then adipocytes and genetic components present in the tissue were removed to obtain a pure extracellular matrix by decellularization. Specifically, the dehydrated tissue was added to a 0.1% sodium dodecyl sulfate (SDS) solution at a rate of 10 g per 1 L, followed by stirring at 100 rpm for 24 hours. Subsequently, the resultant was washed five times with triple-distilled water at 100 rpm for 30 minutes, and 200 ml of DNase having a concentration of 200 U/ml was added thereto, followed by stirring at 37° C. and 100 rpm for 24 hours. Then, the resultant was washed five times with triple-distilled water at 100 rpm for 30 minutes and dried. The decellularized extracellular matrix was freeze-pulverized to a size of about 50 μm by using a freezer mill to obtain powder.

### 2-2. Preparation of Human Skin-Derived Extracellular Matrix

An extracellular matrix was prepared in the same manner as in Preparation Example 2-1, except that skin tissue of a subject was used instead of human adipose tissue.

### 2-3. Preparation of Porcine Kidney-Derived Extracellular Matrix

An extracellular matrix was prepared in the same manner as in Preparation Example 2-1, except that porcine kidney tissue was used instead of human adipose tissue.

### 2-4. Preparation of Mouse Kidney-Derived Extracellular Matrix

An extracellular matrix was prepared in the same manner as in Preparation Example 2-1, except that mouse kidney tissue was used instead of human adipose tissue.

### Preparation Example 3. Zinc Oxide Particles Surface-Modified with Lactide

Zinc oxide (ZnO) nanoparticles surface-modified with lactide were prepared. Specifically, 80 wt % of zinc oxide and 20 wt % of lactide were mixed and stirred for 16 hours in an organic reactor set to 150° C. under a vacuum atmosphere. Subsequently, the stirred solution was dispersed in an organic solvent and filtered through a filter to prepare zinc oxide particles having a particle size of 80 nm and stably dispersed for 3 weeks.

### Preparation Example 4. Kidney Regeneration-Inducing Drug for Preparation of Biodegradable Polymer Scaffold

In the present disclosure, edaravone having a molecular weight of 174.2 kDa was used as a kidney regeneration-inducing drug for use in the preparation of a biodegradable scaffold for kidney regeneration.

### Preparation Example 5. Preparation and Isolation of Exosomes that Secrete Stem Cell Recruitment-Inducing Factor

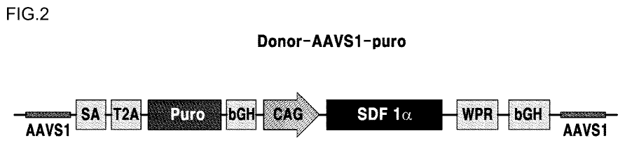

To construct SDF-1α tonsil-derived mesenchymal stem cells (TMSCs), mRNA CRISPR/Cas9 (CosmogeneTech) targeting the safe harbor sites of adeno-associated virus integration site 1 (AAVS1), and the AAVS1 target region of SDF-1α: 5′-CTCCACCCCACAGTGGGGCCACTAGGGGCAGGA-3′ (SEQ ID NO: 1) were transfected into AAVS1. Nucleofection was performed under the following conditions using an SDF-1α sequence (used in the form of a donor vector of FIG. 2) and transfection substrates. TMSCs were seeded onto culture dishes and then stabilized in a 5% CO₂ incubator at 37° C. Subsequently, the TMSCs were cultured, and the supernatant was collected four times at 24-hour intervals. The culture medium was filtered through a 0.2 μm filter to remove impurities, and exosomes were selectively separated and concentrated by using a MWCO 500 kDa filter and a tangential flow filtration (TFF) device.

### EXAMPLES

### Example 1. Polymer Scaffold (1) Containing Kidney Regeneration-Inducing Drug and Extracellular Vesicles that Secrete Stem Cell Recruitment-Inducing Factor

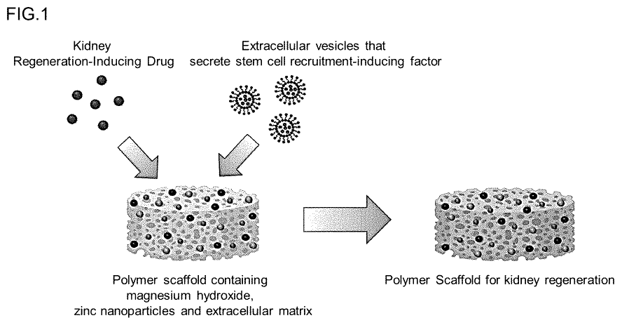

With reference to FIG. 1, a polymer scaffold containing a kidney regeneration-inducing drug and extracellular vesicles that secrete a stem cell recruitment-inducing factor was prepared. Specifically, a polymer solution was prepared by mixing, in an organic solvent, 55 wt % of polylactide-co-glycolide (PLGA) (50:50, molecular weight: 40,000 Da), 11 wt % of the surface-modified magnesium hydroxide particles of Preparation Example 1-3, 28 wt % of the human adipose tissue-derived extracellular matrix of Preparation Example 2-1, 5.5 wt % of the zinc particles of Preparation Example 3, and 0.5 wt % of edaravone (EDV) of Preparation Example 4, with respect to the total weight. Subsequently, the polymer solution was uniformly mixed with 100 to 200 μm of ice particles, followed by freeze-drying for 48 hours using a Teflon mold to prepare a porous scaffold. The porous scaffold was immersed in 70% ethanol for sterilization, washed with sterile distilled water to remove ethanol, and then immersed in a physiological saline solution for hydration. The amount of the exosomes isolated in Preparation Example 5 was measured by a nanoparticle tracking analysis (NTA) method, and then 1×10⁹ of exosome particles were loaded onto the porous polymer scaffold by a simple loading method to prepare a polymer scaffold containing the kidney regeneration-inducing drug.

### Example 2. Polymer Scaffold (2) Containing Kidney Regeneration-Inducing Drug and Extracellular Vesicles that Secrete Stem Cell Recruitment-Inducing Factor

A polymer scaffold for bone regeneration was prepared in the same manner as in Example 1, except that polylactide-co-glycolide (PLGA) (50:50, molecular weight: 110,000 Da) was used instead of polylactide-co-glycolide (PLGA) (50:50, molecular weight: 40,000 Da).

### Example 3. Polymer Scaffold (3) Containing Kidney Regeneration-Inducing Drug and Extracellular Vesicles that Secrete Stem Cell Recruitment-Inducing Factor

A polymer scaffold for bone regeneration was prepared in the same manner as in Example 1, except that polylactide-co-glycolide (PLGA) (75:25, molecular weight: 110,000 Da) was used instead of polylactide-co-glycolide (PLGA) (50:50, molecular weight: 40,000 Da).

### COMPARATIVE EXAMPLES

### Comparative Example 1. Polymer Scaffold

A polymer scaffold was prepared in the same manner as in Example 1, except that only 40K polylactide-co-glycolide (50:50) biodegradable polymer was used, without using surface-modified magnesium hydroxide particles, an extracellular matrix, zinc particles, and edaravone (EDV).

### Comparative Example 2. Polymer Scaffold not Including Edaravone as Kidney Regeneration-Inducing Drug

A polymer scaffold was prepared in the same manner as in Example 1, except that edaravone (EDV) was not used, and 55 wt % of polylactide-co-glycolide (PLGA) (50:50, molecular weight: 40,000 Da), 11 wt % of the surface-modified magnesium hydroxide particles of Preparation Example 1-3, 28 wt % of the human adipose-derived extracellular matrix of Preparation Example 2-1, and 5.5 wt % of the zinc particles of Preparation Example 3 were used.

### Comparative Example 3. Polymer Scaffold (3) Including Edaravone as Kidney Regeneration-Inducing Drug

A polymer scaffold was prepared in the same manner as in Example 1, except that extracellular vesicles (exosomes) that secrete a stem cell recruitment-inducing factor were not used (loaded), and 55 wt % of polylactide-co-glycolide (PLGA) (50:50, molecular weight: 40,000 Da), 11 wt % of the surface-modified magnesium hydroxide particles of Preparation Example 1-3, 28 wt % of the human adipose-derived extracellular matrix of Preparation Example 2-1, 5.5 wt % of the zinc particles of Preparation Example 3, and 0.5 wt % of edaravone (EDV) of Preparation Example 4 were used.

### EXPERIMENTAL EXAMPLES

### Experimental Example 1. Characterization of SDF-1α-MSC

The characteristics of SDF-1α-secreting mesenchymal stem cells (SDF-1α-MSCs) constructed in Preparation Example 5 were analyzed.

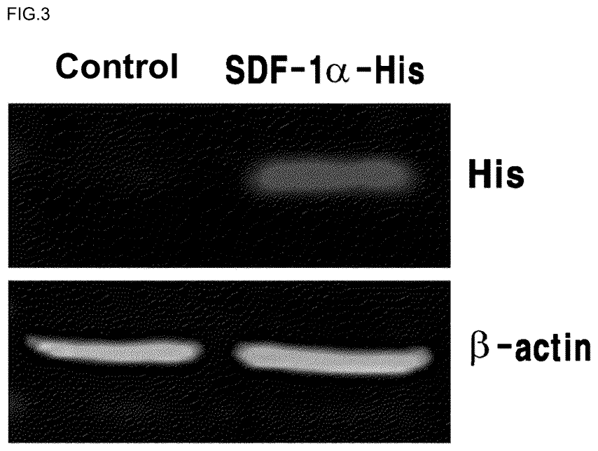

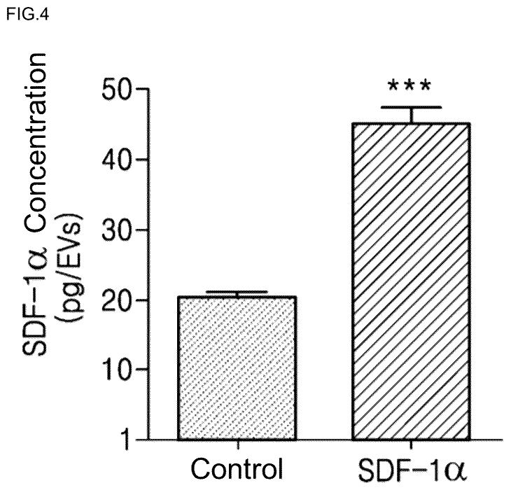

First, as described above, the CRISPR/Cas9 system was used to induce mesenchymal stem cells to secrete SDF-1α, and SDF-1α was used after being labeled with His (see FIG. 2). The expression (presence) of SDF-1α loaded inside the cells and exosomes of SDF-1α-MSCs was confirmed by immunoblotting and ELISA, and the results thereof are shown in FIGS. 3 and 4.

FIG. 3 illustrates the immunoblotting results showing the expression level of SDF-1α-His-conjugated His in the cell lysate of SDF-1α-MSCs, from which the expression of SDF-1α was confirmed. FIG. 4 illustrates the results of analyzing the amount of SDF-1α secreted from exosomes of SDF-1α-MSCs and MSCs by ELISA, from which the amount of SDF-1α secreted from exosomes of SDF-1α-MSCs was 2.5 times greater than that secreted from the exosomes of MSCs.

### Experimental Example 2. Identification of Pore Size and Structure of Polymer Scaffold

Pore sizes and structures of the polymer scaffolds prepared in Example 1 and Comparative Examples 2 and 3 were analyzed by using a scanning electron microscope.

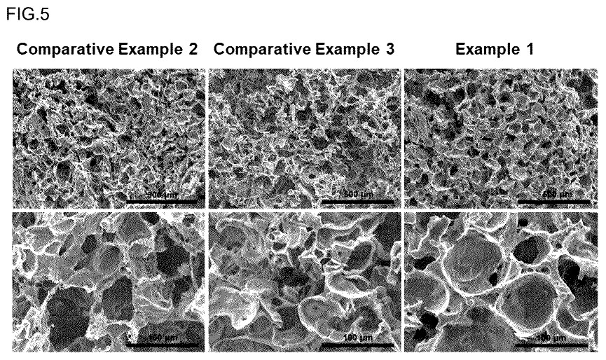

FIG. 5 illustrates scanning electron microscope images acquired by analyzing the pore size and structure of a polymer scaffold according to an aspect. As a result, as shown in FIG. 5, the average size of pores in the scaffold of Example 1 was measured to be 50 μm.

That is, it can be seen that the pore size and structure of the polymer scaffold according to an aspect can be controlled by using the size and content of a porogen.

### Experimental Example 3. In Vitro Wound Healing Migration Assay

To confirm the in vitro wound healing potency of a biodegradable polymer scaffold, the migration enhancement effect of human kidney-2 (HK-2) cells was evaluated. Specifically, the polymer scaffolds prepared in Example 1 and Comparative Examples 2 and 3 were cultured by using HK-2 and transwells for 24 hours, and then opened wound gaps were measured.

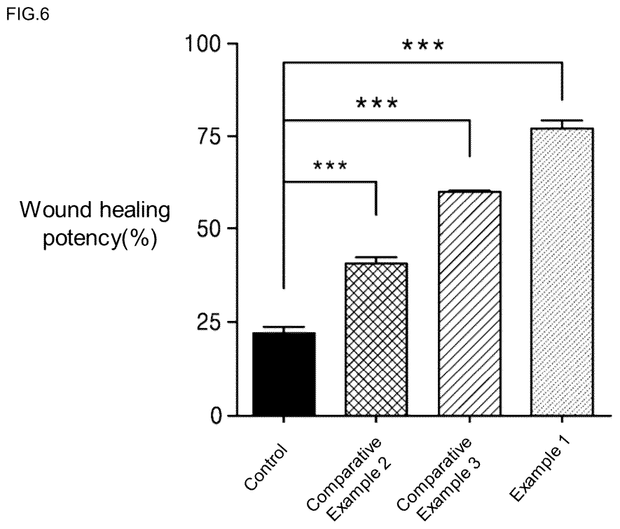

FIG. 6 illustrates the effect of a polymer scaffold according to an aspect on in vitro wound healing, specifically, the results of measuring a wound distance by using ImageJ software.

As a result, as shown in Table 1, it was confirmed that the wound healing potency of Example 1 was superior to those of Comparative Examples 2 and 3.

TABLE 1

| | | Comparative | Comparative | |
|---|---|---|---|---|
| | Control | Example 2 | Example 3 | Example 1 |
| Wound healing | 21.8 ± | 40.6 ± | 60.0 ± | 77.5 ± |
| potency | 1.96% | 2.07% | 0.21% | 2.37% |

That is, it can be seen that the polymer scaffold according to an aspect can promote in vitro wound healing by edaravone and extracellular vesicles that secrete a stem cell recruitment-inducing factor.

### Experimental Example 4. Confirmation of In Vitro Blood Vessel Formation

To confirm the in vitro blood vessel formation of a polymer scaffold, the degree of blood vessel formation of human umbilical vein endothelial cells (HUVECs) was evaluated.

Specifically, HUVECs were dispensed into wells coated with matrigel, the polymer scaffolds prepared in Example 1 and Comparative Examples 2 and 3 were cultured by using transwells for 24 hours, and then the degree of blood vessel formation was measured.

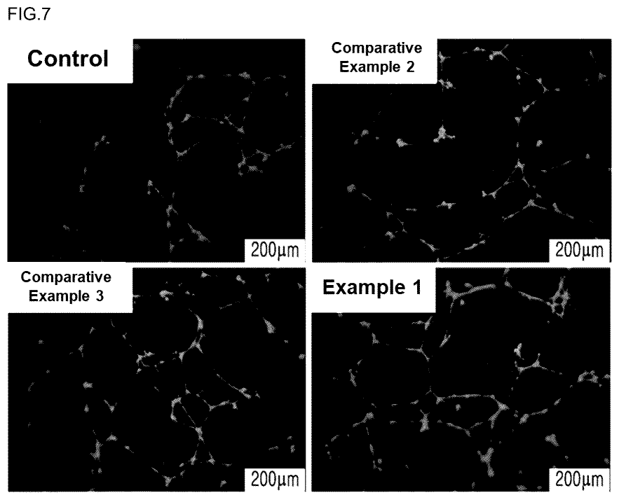

FIG. 7 illustrates the effect of the polymer scaffold according to an aspect on in vitro blood vessel formation, specifically showing the results of observing the patterns of branch points and tubule lengths after staining with Calcein-AM. As a result, as shown in FIG. 7, it was confirmed that the in vitro blood vessel formation ability of Example 1 is superior to those of Comparative Examples 2 and 3.

That is, it can be seen that the polymer scaffold according to an aspect can promote in vitro blood vessel formation by edaravone and extracellular vesicles that secrete a stem cell recruitment-inducing factor.

1. A biodegradable polymer scaffold for kidney regeneration, comprising basic ceramic particles, an extracellular matrix, zinc particles, a kidney regeneration-inducing material, and a biodegradable polymer.

2. The biodegradable polymer scaffold of claim 1, wherein the basic ceramic particles are one or more selected from the group consisting of an alkali metal, an oxide of the alkali metal, a hydroxide of the alkali metal, an alkaline earth metal, an oxide of the alkaline earth metal, and a hydroxide of the alkaline earth metal.

3. The biodegradable polymer scaffold of claim 2, wherein the oxide of the alkali metal, the hydroxide of the alkali metal, the oxide of the alkaline earth metal, or the hydroxide of the alkaline earth metal is selected from the group consisting of lithium hydroxide, beryllium hydroxide, sodium hydroxide, magnesium hydroxide, potassium hydroxide, calcium hydroxide, rubidium hydroxide, strontium hydroxide, barium hydroxide, cesium hydroxide, francium hydroxide, radium hydroxide, magnesium oxide, sodium oxide, lithium oxide, sodium oxide, manganese oxide, potassium oxide, calcium oxide, barium oxide, cesium oxide, and radium oxide.

4. The biodegradable polymer scaffold of claim 1, wherein the basic ceramic particles are surface-modified with a fatty acid, a polymer material, or a mixture thereof.

5. The biodegradable polymer scaffold of claim 1, wherein the basic ceramic particles have a diameter of 1 nm to 1 mm.

6. The biodegradable polymer scaffold of claim 1, wherein the extracellular matrix is isolated from a human body or an animal.

7. The biodegradable polymer scaffold of claim 1, wherein the zinc particles are one or more selected from the group consisting of zinc oxide, zinc sulfide, zinc nitrate, zinc selenide, zinc telluride, zinc nitride, zinc phosphide, zinc arsenide, zinc antimonide, zinc peroxide, zinc hydride, zinc oxalate dihydrate, zinc chloride, zinc bromide, zinc iodide, zinc hydroxide, zinc chlorate, zinc sulfate, zinc phosphate, zinc molybdate, zinc cyanide, zinc metaarsenite, zinc arsenate octahydrate, zinc chromate, zinc pyrithione, and zinc acetate.

8. The biodegradable polymer scaffold of claim 1, wherein the zinc particles are surface-modified with a fatty acid, a polymer material, or a mixture thereof.

9. The biodegradable polymer scaffold of claim 1, wherein the zinc particles have a diameter of 10 nm to 1 mm.

10. The biodegradable polymer scaffold of claim 1, wherein the kidney regeneration-inducing material is edaravone (EDV), extracellular vesicles, or a mixture thereof.

11. The biodegradable polymer scaffold of claim 10, wherein the extracellular vesicles secrete a stem cell recruitment-inducing factor.

12. The biodegradable polymer scaffold of claim 10, wherein the extracellular vesicles are isolated from stem cells derived from one or more selected from the group consisting of umbilical cord, umbilical cord blood, bone marrow, fat, muscle, skin, amniotic membrane, and placenta.

13. The biodegradable polymer scaffold of claim 12, wherein the stem cells are cells genetically engineered to overexpress a stem cell recruitment-inducing factor compared to parent cells.

14. The biodegradable polymer scaffold of claim 13, wherein the stem cell recruitment-inducing factor is stromal derived factor-1α (SDF-1α).

15. The biodegradable polymer scaffold of claim 1, wherein the biodegradable polymer is one or more selected from the group consisting of polylactide, polyglycolide, polycaprolactone, polylactide-co-glycolide, polylactide-co-caprolactone, polyglycolide-co-caprolactone, polydioxanone, polytrimethylene carbonate, polyglycolide-co-dioxanone, polyamide ester, polypeptide, polyorthoester, polymaleic acid, polyanhydride, polysebacic anhydride, polyhydroxyalkanoate, polyhydroxybutyrate, and polycyanoacrylate.

16. The biodegradable polymer scaffold of claim 1, wherein the biodegradable polymer scaffold comprises 1 to 20 wt % of the basic ceramic particles, 10 to 50 wt % of the extracellular matrix, 1 to 10 wt % of the zinc particles, and 25 to 85 wt % of the biodegradable polymer with respect to a total weight of the biodegradable polymer scaffold.

17. The biodegradable polymer scaffold of claim 16, wherein, in case that the kidney regeneration-inducing material comprises edaravone, the biodegradable polymer scaffold comprises 0.1 to 1 wt % of the edaravone with respect to the total weight of the biodegradable polymer scaffold.

18. The biodegradable polymer scaffold of claim 16, wherein, in case that the kidney regeneration-inducing material comprises extracellular vesicles, 1×10⁶ to 1×10¹² of the extracellular vesicles are comprised in the biodegradable polymer scaffold.

19. A bioimplant for kidney regeneration, comprising the biodegradable polymer scaffold of claim 1.

20. A method of preparing a biodegradable polymer scaffold for kidney regeneration, the method comprising: preparing a first polymer solution by mixing basic ceramic particles, an extracellular matrix, zinc particles, and a biodegradable polymer;
preparing a second polymer solution by mixing the first polymer solution and a porogen; and
drying the second polymer solution to prepare a porous scaffold, and the method further comprising:
in the preparation of the first polymer solution, mixing together with a kidney regeneration-inducing material; or
after the preparation of the porous scaffold, loading the kidney regeneration-inducing material.
